(19) United States
(12) Patent Application Publication
(10) Pub. No.: US 2022/0203168 A1
(43) Pub. Date: Jun. 30, 2022

(54) Systems and Methods for Enhancing Exercise Instruction, Tracking and Motivation

(71) Applicant: Nicholas calderon, San Juan Capistrano, CA (US)

(72) Inventor: Nicholas calderon, San Juan Capistrano, CA (US)

(73) Assignee: Veki, Inc., San Juan Capistrano, CA (US)

(21) Appl. No.: 17/565,447

(22) Filed: Dec. 29, 2021

Related U.S. Application Data
(60) Provisional application No. 63/131,678, filed on Dec. 29, 2020.

(51) Int. Cl. A63B 24/00

(52) U.S. Cl. CPC A63B 24/0062; A63B 24/0006; A63B 24/0075; A63B 2220/05; A63B 2024/0068; A63B 2024/0012; A63B 2024/0096; A63B 24/0087

(57) ABSTRACT

Systems and methods for enhancing fitness instruction, performance tracking, and motivation is described. A device collects information about an exercise movement and predicts movement of a subject in the third dimension. A fitness instructor performing exercise movements, and a user performing exercise movements, can be used to train a predictive model. A predictive model can suggest exercise feedback, measure performance of an exercise movement, and motivate a user to exercise. The power generated by a user can be measured. Cryptographic hashing and a distributed ledger network can be used to enhance exercise motivation and provide rewards for completing exercises movements. Rewards may be registered on a distributed ledger network and become the property of a user.

The present application claims the benefit of Provisional Application No. 6313167 filed Dec. 29, 2020, entitled “Enhancing Fitness Instruction, Feedback, Performance Tracking, and Motivation.”

### FIELD OF THE INVENTION

The present technology relates to fitness and more specifically enhanced exercise instruction, tracking and motivation.

### BACKGROUND

For exercise instruction, a person can hire a trainer or coach, attend group classes, study literature, or watch instructional videos. There are several setbacks to these methods. A personal trainer or coach can cost hundreds of dollars an hour, attending group classes provides limited instructor to trainee interaction, studying literature takes a lot of time and preparation outside of exercising, and instructional videos provide no feedback or instructor interaction.

Motivation is one of the largest problems in exercise. A personal trainer or coach can help motivate a person to exercise more frequently, better, or with higher effort. But for many, hiring a personal trainer is cost prohibitive. It can require building an intimate relationship with a stranger, which may cause discomfort from exposer to unwanted attention. Finding an exercise partner can provide motivational benefits. However, the hurtles to finding an exercise partner can be daunting. In addition to overcoming possible discomfort from unwanted attention, fitness partners often require finding a person with a compatible personality, who has a similar fitness level, and that has a compatible schedule.

### SUMMARY OF THE INVENTION

The present invention enhances exercise instruction, performance tracking and motivation. Embodiments of the present technology comprise a novel method for estimating the third dimension (3D) of a human pose, improving upon systems and methods that rely on depth sensors or computationally heavy physics engines. In one embodiment, a system for enhancing fitness instruction and motivation comprises an exercise device wherein a video camera, data sever, and computer connects to a user display and can be configured to track a user and compare a user movement to an instructional movement to compute and communicate instructions to the user through a user display. Embodiments of the novel technology presented can instruct a user on how to perform an exercise movement, personalize instructions, provide real-time feedback, and learn how to adjust instruction based on how a user is performing, has performed in the past or on how other users performed in the past. It can act as a robust performance tracking device, making speed, cost, and accuracy improvements over solutions that rely on depth sensors. It can track performance with a continuous scoring method comprising novel methods of measuring the intensity of exercise performance and form correctness. It can measure the power a user exerts during an exercise movement. It can provide robust rewards that can become a user's property, comprising a cryptographic hashing function, consensus validation method and distributed ledger network, to motivate users to meet fitness goals, increase performance, or workout frequency without the need for the physical presence of a fitness partner, trainer, or coach. It can reward users with assets registered on a distributed ledger network that can become the property of a user. It that can be embodied in a portable device, used with various display units, and is not dependent on a single display unit.

### DETAILED DESCRIPTION OF THE DRAWINGS

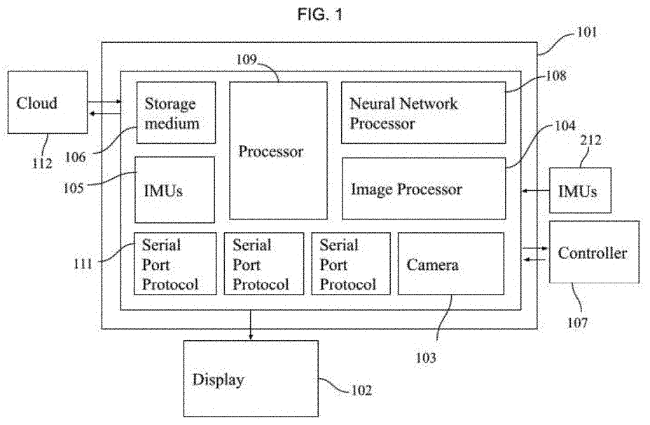

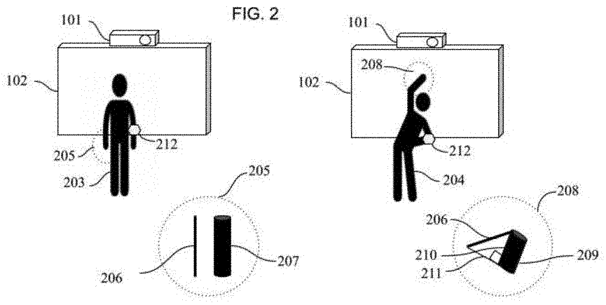

FIG. 1 shows a block diagram of components of a computer vision exercise device exemplary in FIGS. 2, 3, 4, 5, 6, 7, and 12.

FIG. 2 shows an embodiment of a process for predicting the third dimension of body features with monocular vision.

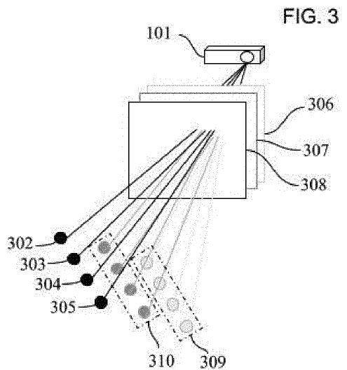

FIG. 3 shows a process of correcting feature estimation with monocular vision and sensors readings across sequential images.

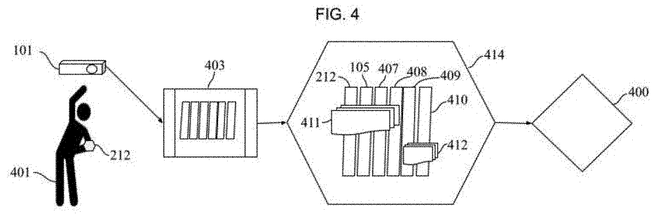

FIG. 4 shows a flow of information in training a predictive exercise model.

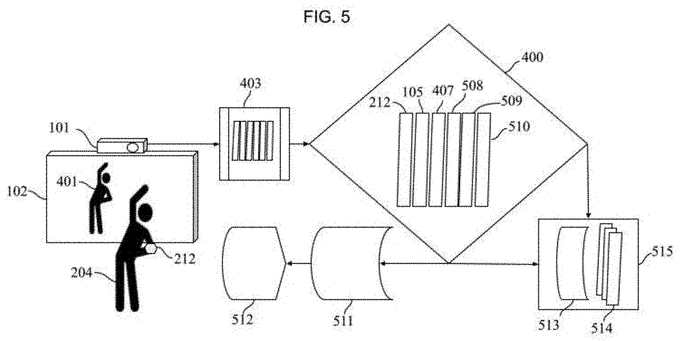

FIG. 5 shows a flow of information of an embodiment for predicting exercise feedback, instructions, performance tracking, and motivation.

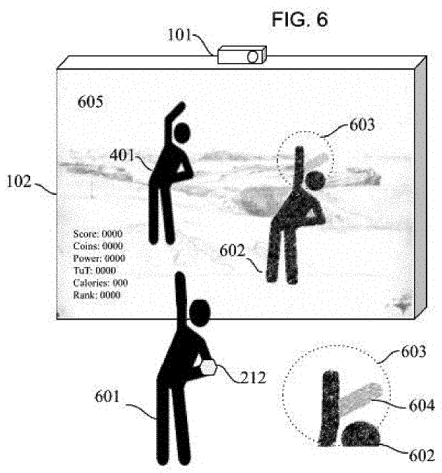

FIG. 6 shows an exemplary third person view of a user interacting with a virtual environment projected on a television with a computer vision device.

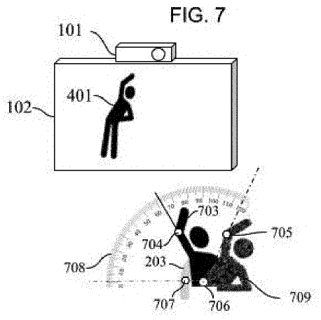

FIG. 7 shows an exemplary method for how intensity and form of an exercise movement can be scored on a continuum computing angles amongst features in accordance with embodiments of the present invention.

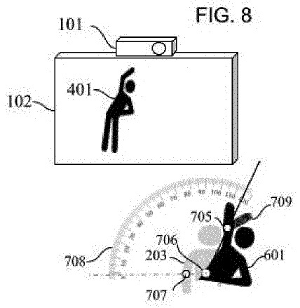

FIG. 8 shows an exemplary method for how intensity and form of an exercise movement can be scored on a continuum computing angles amongst features in accordance with embodiments of the present invention.

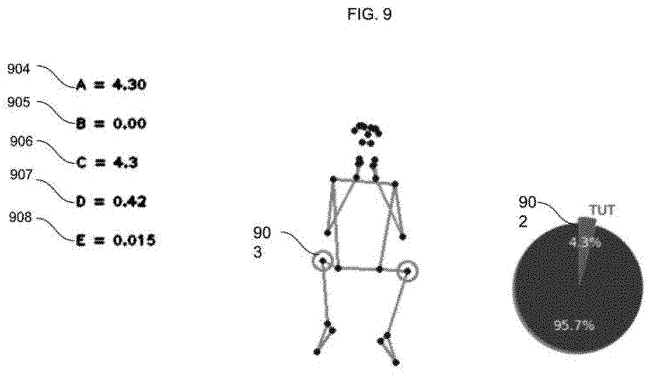

FIG. 9 shows an exemplary method for how exercise performance tracking features can be computed in accordance with embodiments of the present invention.

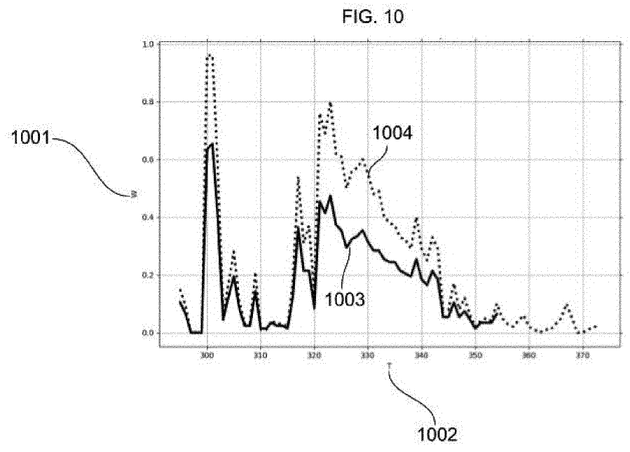

FIG. 10 shows an exemplary method for how power generated by a user when performing an exercise movement can be tracked in relation to the power generated by a fitness instructor.

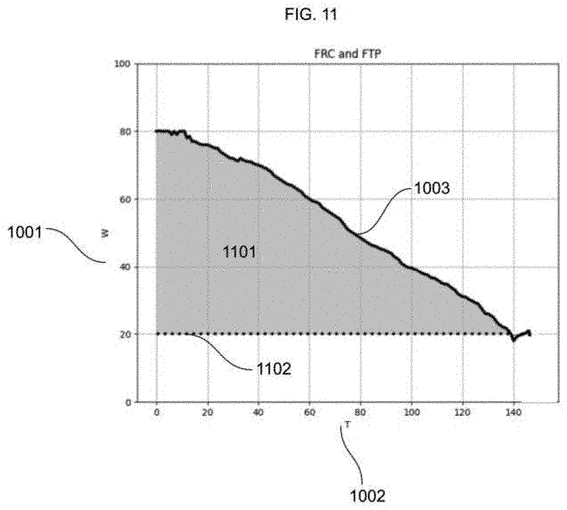

FIG. 11 shows an exemplary method for how functional reserve capacity and function total power can be tracked throughout an exercise to in accordance to an embodiment of the present invention.

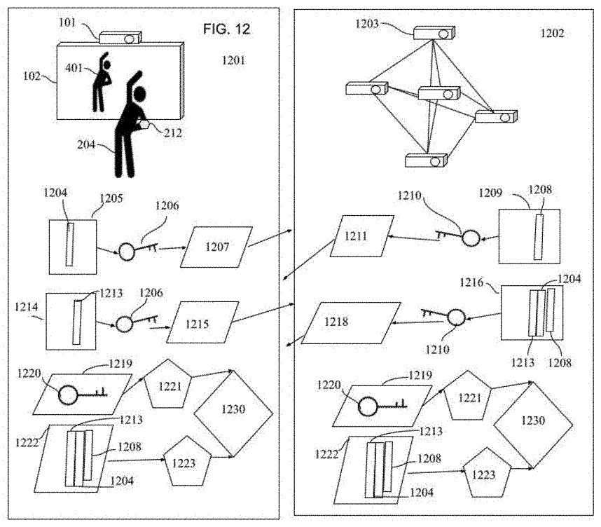

FIG. 12 shows a flow of information for a distributed ledger network to generate a coin reward for a user performing at least one exercise movement in accordance to one embodiment of the present invention.

### DETAILED DESCRIPTION

Embodiments of the present technology fulfill a need for a device to enhance exercise instruction, performance tracking, and motivation. Systems and methods for enhancing exercise instruction, performance tracking, and motivation, without the need for the physical presence of a personal trainer, coach, or fitness partner, and improving on systems and methods comprising computational heavy physics engines or depth sensors, that satisfy a need a user has to learn how to exercise better, gain insights into their progress, improve their health and fitness, and be motivated to exercise, are presented. Such a device would open exercise instruction to a wide range of people, provide a more robust method to instruct exercise, and increase the chances of exercise providing fitness benefits. Such a device would reduce the chances of injury and improve health, which would lower healthcare costs.

Embodiments of the present technology, comprising computer vision device (101), can be used to enhance exercise instruction, tracking and motivation. A computer vision device (101) can communicate information to a user through a general-purpose display (102). A video camera (103) can be coupled to a device to capture a user movement. A video camera can be fitted with a wide field of view lens. A camera lens may be hidden within an enclosure and a material may be placed in front of a camera lens so that when the camera is in operation, the camera is not visible to a user performing an exercise movement. An enclosure around the device, comprising a hinge fitted to the bottom of the enclosure, can be designed to be placed on a table in front of a display (e.g., television) or mounted to on top of a display. When a hinge fitted to the bottom of a device is closed, it acts as a stand for the device to be placed on a flat surface. When a hinge is open, it can be mounted on top of a display (e.g., television), the hinge, with friction, torque, or spring resistance, can hold the device on the back of a display at an angle pointing up, or down, as may be configured by a user. Internal Measurement Units (IMUs) (105) can be contained within the device to inform a computer of a device (103) orientation as it relates to the field of view of a camera within the device. IMUs (110) coupled to the device, outside of a device enclosure, can be attached to a user, communicating information to a serial port protocol (111) within a device about the orientation and movement of one or more coordinates on a user body. One or more processors (104, 108, 109) and a computer readable storage medium (106) may be coupled to the device, wherein the computer, coupled to internal (105) and external IMUs (212) that can be worn by a user, can be configured to perform human pose estimation on images from at least one image from a camera. A processor (104) can normalize images from a video camera (103) to reduce lens distortion, warping, or barreling effects. A processor (104) can be coupled to the device to resize images to reduce the amount of data for processing. It may reduce the amount of data by combining software methods, such as detecting the user, segmenting the user from the image, cropping the image around the user, can be used. A computer-readable storage medium (106) comprising executable instructions can compare a user executing an exercise movement (204) with at least one pose of a fitness instructor executing an exercise movement (401), wherein the distance can be measured between one or more features on a user pose and the distance of one or more features on an instructor pose (FIG. 5, FIG. 7 FIG. 8). At least one neural network processor (108) can be used to accelerate machine learning operations (e.g., human pose estimation, pose tracking, person detection, image segmentation, etc). A computer vision device can receive information from a cloud (112) (e.g., data server or network of computer vision devices serving as a data server to nodes receiving as a receiver). A cloud (112) can store exercise instructions, exercise videos, feedback instructions, and pose information for exercises. A data sever (112), comprising one or more computer vision devices, can be used to facilitate the storage and transfer of exercise information, such as exercise instructions and records, and can be used as a distributed ledger network. A controller (107) coupled to the device, wherein the controller can be used to navigate through application interface pages displayed on a display (102) or a smartphone application can be used as a remote control (107) to navigate through the pages of a device application shown on a display (102). A serial port protocol (111) can connect a remote, external IMUs, and a display to a computer vision device.

Embodiments of the present technology, comprising systems and methods to augment an exercise video, can improve exercise instruction with simulations. A computer vision device (101) can project an exercising training video onto a display. An exercise environment comprising an exercise setting (e.g., a fitness studio, gym, field, beach, animated scene, etc.) with one or more instructors (i.e., an instructor in a pre-recorded video, live video, animated video, or still images), can be augmented with a simulation of one or more users (605). Through one or more software applications and a computer vision device, a simulated representation of a user (602) can be created. A simulated representation can represent a similar likeness to a user, fitness instructor, generic human figure, or animated figure. A simulated representation of a user can be an asset that is property of a user, such as a non-fungible token. As a user stands in front of a computer vision device and performs an exercise movement, a user movement can be capture by one or more sensors (204). Human pose estimation can be computed. Features of a 3D pose can be estimated (205). Estimated features of a user can be communicated to a user interface application (602). Through one or more software application, a user body movement in front of a computer vision device can control the movement of a 3D model of a user within an exercise environment projected onto a display (601). Performance tracking and feedback information can be presented to a user in a virtual environment through a dashboard. If a user performs an exercise movement incorrectly (603), a simulated representation of a user (604) can demonstrate to a user, correct form. For example, if when performing a squat, a user's legs are identified as being too far apart, a virtual simulation can display a virtual representation of a user with legs outlined in red. An instructional silhouette (604) can be overlayed onto a user virtual simulation (603) showing an animation of where a user's legs should be placed. When a user moves their legs to align with the instructional silhouette, the virtual simulation of a user's legs can move to align with the instructional movement, a red error outline can disappear, a green outline can appear momentarily to signal the user is now in the correct position, and an instructional silhouette can disappear. In another example, if performing a squat, a user's legs are identified as being too far apart, a 3D model can display a virtual representation of a user's legs highlighted in red. A simulated representation of a user (602) can move independently of a user to demonstrate how far a user's legs should move in order to be placed in the right position, while a virtual user silhouette can continue to follow a user movement to show where the user's legs are positioned. If a user moves to follow the virtual instruction, a user silhouette can move to follow a user movement, if a user aligned with the simulated representation of a user, a silhouette can disappear.

Embodiments of the present technology, comprising systems and methods for estimating the 3D position of features on the human body, can enhance exercise instruction, feedback, performance tracking and motivation. Embodiments of the present technology, comprising a computer vision device, machine learning and kinematics, can estimate the 3D movement of body features (e.g., the depth distance body parts move, head, eyes, feet, feet ankle, hands, etc.) form monocular images of an exercise movement (FIG. 2). Embodiments of the present technology, comprising a computer vision device, machine learning, and algorithms, can estimate the 3D position of body features monocular images from a video of an exercise movement, improving on the speed, accuracy, and cost of technologies comprising depth sensors or computationally heavy physics engines.

Embodies of the present technology can be used to record an exercise movement. 2D pose estimation can be performed on the instructional movement. Pose estimation can classify, predict, and track features on the human body (e.g., eyes, forehead, brow, head, mouth, neck, torso, limbs, joints, feet, hands, fingers, etc.). Pose estimation can be trained to classify, predict, and track characteristics about the body being observed (such as state e.g., standing, lying down, body orientation, limb orientation, etc.) or mood (e.g., happy, sad, bored, tired). When 2D pose estimation is performed, embodiments of the present technology can perform analysis on the video frames to estimate the depth movement of body parts, features, or points on the body, such as joints or limbs.

A computer vision device may be connected to a monitor displaying an instructional video (102) or instructional simulation (FIG. 6). The instructional video may ask a user to face the camera and stand in a neutral, upright posture (203). When the human body is in a neutral, standing posture (203), a baseline posture or body position (206) can be recorded, estimating the dimensions of the body, body parts (205), or distances between feature coordinates estimated on the body from 2D pose estimation. Data can be collected, form user input or from an electronic device where a user inputs data about the dimensions of the human body, to inform a computer about the size of a user body. Data collected about a user physical attributes (e.g., height, weight, etc.) can be used to inform a baseline posture. Data can be collected about a user by a computer vision device (101) to estimate dimensions of the human body, body parts, or distances between features to inform a baseline posture (206). As the body moves from the baseline posture (204), an algorithm comprising 2D pose estimation, kinematics, user orientation with the respect to the camera, and geometry can be used to estimate depth of features on the body in relation to the body or the body's distance from a monocular sensor (208).

2D pose estimation can be performed on a user. It can predict body orientation with respect to a camera and state. It can measure the distance amongst body features (206). It can measure the distance of features with respect to the distance amongst other features. Data can be collected from a user (e.g., entered into a computer by a user) about a user height, weight, or body dimensions. A software application can be run that takes measurements of a user, about a user height, weight, or body dimensions. For example, a user can be asked about their height. A software application can ask a user to move in a certain way so that that a computer vision algorithm can measure the size and dimesons of body features. When the body is in a baseline posture (203), one or more software applications can measure the distance from left ankle to left knee, from left knee to left hip, from left hip to left shoulder, from left shoulder to left eye, from left eye to the top of the head. The distance amongst these points, along with the camera angle through which images are captured and 2D pose estimation is performed, may be computed to estimate the total dimensions of a body or to estimate a ratio for dimensions of each body part in relation to the total dimensions of a body. Key features or feature points can be selected to compute angles. Data collected can classify two-dimensional attributes and dimensions of body features in observed states with relation to a camera, such as a user baseline posture. A computer can be programed to save these attributes for a user, such as storing a baseline posture.

Embodiments of the present technology, comprising an instructional video, may ask a user to perform an exercise. 2D pose estimation may be performed as a user is in a baseline posture (203). At least one dimension of at least one body feature (207) can be measured, such as length (206), in a baseline posture (203). As a user performs an exercise (204), 2D pose estimation can be performed. At least one dimension of at least one body feature (209), such as length (210), can be measured. If the distance of at least one dimensions, such as the length, is different in a baseline posture (206) as compared to during an exercise movement (2010), than depth (211) can be estimated. For example, if an instructional video can ask a user to stand, facing a camera, and perform a squat. When a user is standing in a baseline posture, the distance from the left ankle to the left knee can be measured as (c), when in a downward squatting position, the distance from the left ankle to left knee can be measured as (b). The change in perceived size of the left ankle to left knee can be measured as (a). The formula a equals the square root of c\hat{ }2−b\hat{ }2 can estimate the depth of which the knee has moved. Kinematics can inform the knee has moved forward by the approximate distance of a.

Embodiments of the present technology can normalize a baseline posture or camera images from distortion introduced by lens composition (i.e., barrel effects from a wide field of view) and camera orientation (i.e., vertical offset, horizontal offset, distance, pitch angle, yaw angle, role angle,) with respect to a user position. The embodiments presented of the present technology presents opportunities to enhance fitness instruction with monocular vision. For example, estimating the depth movement of the left ankle to left knee, combined with the left knee to the left hip, can be used to estimate the depth movement of the left hip in relation to the knee or ankle point. The same calculations can be performed as an instructor performs the exercise movement. The difference in the placement of the hip in relation to the knee or ankle point between the user and instructor can be measured to determine how closely a user form is in respect to the form of an instructor. The method presents a solution for predicting depth of body movement with greater accuracy, improved speed and efficiency, and lower computational costs than prevailing methods comprising depth sensors, computationally heavy physics engines, or larger neural networks perform for exercise movements with less speed, accuracy, and efficiency of the present technology.

Embodiments of the present technology, comprising a human pose estimation correction model, can improve depth estimation with physical sensors. Data from at least one IMU (212) within a wearable device (e.g., accelerometer, gyroscope, etc.) can be used. A user can wear a device (e.g., on their wrist, arm, chest, leg, etc.) that contains IMUs (212). A computer vision device can be programed to detect and track the position of the device worn by a user (101). A computer vision device (101) can contain IMUs (105) to provide information regarding the orientation of the camera (103) within the computer vision device. When a user is in a neutral position (203), a computer vision device (101) can track a point on the human body where a wearable device is worn (212). As a user performs an exercise movement (204), the computer vision device can compute the orientation and movement of the sensor point with monocular vision and the worn IMUs (212) within the wearable device. The orientation and linear distance, as computed by monocular vision and IMUs within the wearable device, can be tracked over a series of frames (FIG. 3) (e.g., when the user is in a neutral position until a user completes an exercise movement) and can be interpolated with the orientation data from the IMUs (105) within a computer vision device (101), presenting a bundle adjustment problem (310, 309, 302-305). The bundle adjustment problem can be solved (e.g., with linear regression, Levenberg-Marquardt algorithm, least-squares method, etc.) to improve the accuracy of the monocular depth estimation method presented in at least one area. It can help reduce distortion introduced by monocular vision and the orientation of a user with respect to a camera (i.e., vertical offset, horizontal offset, distance, pitch angle, yaw angle, role angle). Methods to reduce accumulating measurement error can be used to enhance the accuracy of IMUs readings from IMUs attached to a user (212). When a computer vision device (101) is moved, the orientation of the camera can be changed, triggering one or more software applications to recalibrate a correction model.

Embodiments of the present technology include systems and methods for enhancing exercise feedback. A computer vision device (101) can be placed so that its video camera can record a fitness instructor (FIG. 4) (e.g., exercise instructor, fitness professional, personal trainer, athlete performing, a user performing one or more exercise movements demonstrating proper form; a user performing one or more exercise movements demonstrating improper form). As a fitness instructor performs an exercise movement (401), camera images can be captured. One or more frames from the video can be processed (403) (e.g., normalize, denoise, dewarp, resized, etc.). Data from one or more IMUs worn by a fitness instructor (212) can be labeled for one or more exercise movements (401). Data collected form one or more IMUs (105) within a computer vision device (101) can be labeled (411) for one or more exercise movements (401). IMUs data and image data can be used to correct for distortion introduced by the camera orientation with respect to a user position and orientation. Through one or more software applications, a 3D pose of a movement can be estimated (FIG. 2). One or more exercise movements can be performed by one or more fitness instructors (401) to train a machine learning model to recognize an instructional pattern (FIG. 4). Attributes related to the fitness instructor (409) (e.g., body type, composition, orientation, ability, clothing), environment (410) (e.g., lighting, wall color, background items, clutter, etc.), body movement (408) (e.g., movement speed, direction, intensity), or exercise execution (408) (e.g., correctness, errors, form feedback, a performance score, etc.) can be labeled (411) for one or more exercise movements (401) for one or more fitness instructors. Labels can be organized hierarchically. For example, if there are 10 errors in exercise execution for a given exercise movement, each error can be ordered in importance from 1 to 10. During inference, the ordered structure of the labels can inform how to order feedback to give to a user. A 3D human pose estimation (407) (e.g., feature position, key point position, normalized key point position, or angles amongst one or more sets of features, etc.) can be labeled for one or more exercise movements (414) by one or more fitness instructors. One or more features or angles amongst one or more sets of features (408) can be labeled related to body movement (e.g., movement speed, direction, intensity) and exercise execution (408) (e.g., correctness, errors, form feedback, a performance score, etc.). Data collected can be used to train a predictive model (400) (e.g., neural network, deep learning, etc.) to train a computer vision device (101) to recognize attributes about a subject standing in front of its camera, about the environment (410), body movement (408), and exercise execution (408). Unlabeled data can be fed to a trained machine learning model (FIG. 5.) to predict labels (514) for an exercise movement and can be used to reduce labeling for training a machine learning model (semi-unsupervised learning).

A computer vision device (101) can be connected to a display (102) and can be placed so that its camera can record the movement of a user. A computer can provide exercise instructions to a user through a display (102). As a user performs an exercise movement, pose estimation can be performed. Data from one or more IMUs worn by a user can be collected (212). Data form one or more IMUs within a computer vision device can collected (105). Through one or more software applications and a computer vision device (101), a 3D human pose can be estimated (FIG. 2). Data collected can be processed (403) and fed to a machine learning model (400). A machine learning model (400) can be trained to compare the movement of a user with a fitness instructor to produce feedback. A machine learning model (400) can predict labels (511) for an exercise movement (204). A computer can sort predictions and prioritize feedback (512) to send to a user performing an exercise movement. Feedback can be sent to a user (e.g., by video, audio, graphics, written commands) instructing a user on how to align with the movement of a fitness instructor (e.g. 604) (e.g., adjust intensity, form body or limb position, orientation, speed, force, stability, range of motion, rotation of body parts, etc.). For example, if a user is performing a squat and feet are estimated as too close together, a computer can give feedback to a user to widen their feet.

As a computer performs an exercise movement in front of a computer vision device, data from one or more IMUs worn by a user can be collected. Data form one or more IMUs within a computer vision device can collected. Through one or more software applications and a computer vision device, a 3D human pose can be estimated. A machine learning model can measure one or more features or angles amongst one or more sets of features. Feedback can be provided based on the difference in one or more features or angles when a user performs the exercise movement can be compared to the difference in one or more features or angles when a fitness instructor performs the exercise movement. For example, when performing a squat, if the distance from the left ankle to right ankle relative to body size is half the distance of the left ankle to the right ankle for the fitness instructor, a computer can give feedback to a user to widen their feet.

Embodiments of the present technology are not limited to the present representation. Embodiments of the present technology, comprising a machine learning and mathematical algorithms, can be used to compare an instructional movement to a user movement to compute exercise feedback. For example, for computing feedback, embodiments of the present technology can produce a 3D depth estimation of a user movement. The movement and angles and features of a 3D pose as a fitness instruction performs a fitness movement, with respect to time, can be saved as an instructional pattern. The instructional pattern can be used to train a machine learning model, the instructional pattern can be used to compute feedback, or a combination of the instructional pattern training a model and computing feedback can be combined. For example, an algorithmic approach can compare an instructional pattern with a user movement. A 3D pose can be estimated from 2D images captured by a computer vision device. An instructional pattern can be recorded from an instructional movement, comprising attributes such as angles, angle thresholds and relative distance amongst body features or points with respect to the time it takes to complete an exercise movement. A user exercise movement, as computed with a 3D pose estimation, can be compared to an instructional pattern of angles and threshold values to produce instructional feedback. Attributes such as angles, angle thresholds and relative distance amongst body features or points with respect to the time it takes to complete an exercise movement can be used inform a predictive model, can be used. As those skilled in the art may attest, a blend of the embodiments may be used. Embodiments of the present technology, comprising a computer vision, machine learning and kinematics, enhance instructional feedback with higher speed, accuracy, efficiency, and lower computational expense, availing novel embodiments such as those disclosed herein and such as those embodiments that can be extended from the disclosure by those skilled in the art.

Embodiments of the present technology fulfill a need for a method to evaluate and score exercise movements based on individual ability. The novel scoring method can allow a novice and experienced trainee to exercise to exercise instructions at different difficulty levels and receive useful feedback and scoring based on their respective abilities. It can track the performance of an exercise movement in a continuous method that allows people with varying abilities to draw value from an instructional video which can reduce the need for creating instructional videos at different difficulty level.

Embodiments of the present technology, realized by one or more software applications and a computer vision device, can be used for scoring of an exercise movement on a continuum of intensity or form correctness (FIG. 7, FIG. 8). A computer vision device can be placed so that its video camera can record a fitness instructor. As one or more fitness instructors can perform an exercise movement (401), a video camera images can be captured. One or more images from a video camera can be processed (403). Data from one or more IMUs worn by a fitness instructor can be collected (212). Data form one or more IMUs within a computer vision device can be collected (105). Through one or more software applications and a computer vision device (101), a 3D pose of an exercise movement can be estimated (407). One or more features (e.g., 704) or angles (708) amongst one or more sets of features (704, 705, 706, 707) with respect to time, can be recorded for a body movement, or exercise execution. An instructional baseline or ideal movement pattern (709) can be created for features or angles (705, 706, 707) for a body movement or an exercise movement with respect to the time it takes to complete one repetition or within one time interval. Analysis of one or more fitness instructors can compute a margin of error for an instructional baseline (709), for example, can be recorded in a table. The instructional baseline can be used to evaluate form correctness or intensity of a user with a mathematical algorithm or during inference of a predictive model. For example, a form score can be based on pose estimation feature values that relate to the entire body moving in alignment as exhibited in a fitness instructor or instructional baseline. An intensity score can be based on pose estimation feature values that relate to the maximum inflection point and movement speed as exhibited in a fitness instructor or instructional baseline. Similarly, the furthest point at which a user can move their body from a starting position along a progressive scale of difficulty to a maximum inflection point, can be recorded to provide a score that can be broken down on a continuum (FIG. 7, FIG. 8), or a predictive model could be trained to compute the score.

Embodiments of the present technology, comprising a method to compare a user movement to a fitness instructor, can produce a continuous performance score. Data from one or more IMUs worn by a user can be collected. Data form one or more IMUs within a computer vision device can collected. Through one or more software applications and a computer vision device, a 3D human pose can be estimated. A machine learning model can measure one or more features or angles amongst one or more sets of features, with respect to time. Features or angles with respect to the time it takes to complete one repetition or within a time interval can be compared to a fitness instructor baseline to compute a delta value for form correctness or intensity. The delta value can be computed to produce a score or a predictive model can be trained to a compute a score. For example, if for a squat, the maximum intensity (i.e., the furthest depth position of the squat) were measured by a hip height of 10 and depth of 10, and a user achieved half of the maximum intensity during a squat (i.e., hip height of 5 and depth of 5), a repetition may produce a delta value of 0.5. If a multiplier of 100 is given, or a total score of 100 were possible for the repetition, a user may achieve an intensity score of 0.5*100, or 50. The novel scoring method embodiments can be applied to future workouts or workout planning. For example, if proper form is exhibited for consecutive repetitions or intervals, more difficult exercise variations (e.g., deep squat instead of full squat), routines (e.g., higher repetition count), or more difficult instructional content can be suggested as a result. If poor form is exhibited, easier variations (e.g., half squat instead of a full squat) of an exercise, routines (e.g., lower repetition count), or easier instructional content can be suggested to a user.

A continuous scoring mechanism can enable exercise content to produce higher value across users or user groups with different abilities. For example, if a user is unable to perform with high intensity but can maintain proper for (FIG. 7) (e.g., they cannot perform a deep squat but can perform a full squat with proper form), they can still achieve a perfect form score even if scoring low on intensity. Since form is important in preventing injury and maximizing gains, an intensity score can be discounted in comparison to a form score to encourage better form. Embodiments of the present technology can learn from a user intensity and form score to suggest a workout plan. If a user performs strongly in form and has a goal to increase strength, exercise instructions can be adjusted for a user to recommend higher intensity exercises, exercise variations, or more difficult content.

The present technology allows for a wider range of difficulty to be accessed from a piece of instructional content. It can allow useful feedback linked to a user ability. The embodiments, systems and methods disclosed can allow a user to participate in a hard workout at a novice level without being discouraged since they can perform each exercise movement achieving a low intensity score, a high form score, and a relatively higher overall score. The embodiments, systems and methods disclosed can allow an advanced user to participate in an easy without without being bored since they can be instructed to complete harder exercise variations, achieving a high intensity score, a high form score and a higher overall score. Embodiments of the present invention can enable advances in leaderboard technology, such as allowing cohorts of users at varying levels of ability to compare their performance amongst a peer group, enabling larger workout classes with enhanced leaderboard representations.

Embodiments of the present technology can provide robust performance tracking, improving with speed and efficiency when compared to methods that rely on depth sensors or physics engines to enhance exercise feedback. It can exceed the accuracy of current methods, such as depth sensors, for the purpose of predicting nuanced differences in depth needed to track exercise performance. Embodiments of the present technology can provide robust performance tracking of a user performing an exercise or a series of exercises over time (e.g., time under tension, power exerted, stability, range of motion, rotation of body parts, form, heartrate, or calories burned, movement acceleration, intensity, etc.).

Embodiments of the present technology can compute time under tension (TuT). TuT tracks exercise performance more precisely allowing a user to better understanding how movement relates to fitness levels since TuT is more directly related to protein synthesis as compared to methods commonly used to track performance, such as repetition counting. Embodiments of the present technology, comprising one or more software applications and a computer vision device, can compute time under tension (TuT) for muscles or muscle groups. As a user performs an exercise movement, a 3D human pose can be estimated. Core muscle groups can be identified for each workout. For example, with a squat, the gluteal group can be identified as a core muscle group or the upper legs can be identified as a muscle group (e.g., gluteal muscle group, quadriceps muscle group, biceps femoris, gastrocnemius, peroneal muscle group, etc.) for which TuT is measured. As a human pose is tracked performing a squat, centric and acentric time can be calculated for the selected muscle or muscle groups. If during a squat, the entire leg is selected as the muscle group to measure TuT for (FIG. 9), tension time can be accumulated for the total time when a user is activating the selected muscle groups and subtracting rest time (e.g., if squatting for 3 seconds, a tension time score would accumulate to 3 seconds, if a user takes a break for 2 seconds and starts squatting again at the 5 second mark for 3 seconds, TuT would be 6 seconds). Alternatively, tension time can accumulate for the total time when a user is activating a narrow selection of muscle or muscle group. The present technology is advanced over previous methods since it can replace repetition counters with a tension time counter (902). It can isolate which muscle groups are being targeted. For example, since the present technology can be built on a novel embodiment comprising a computer vision device and a depth estimation of body features to enhance fitness instruction, narrow muscle groups can be tracked for measuring TuT, where a centric and ascent time for isolated muscles or groups of muscles, can be computed.

Embodiments of the present technology, comprising a method to compute power exerted during a movement with computer vision, can enhance exercise feedback. Measuring power has largely been reserved for athletes in competitive cycling. Power exertion and tracking can enhance exercise feedback and tracking by informing how much power may be exert throughout a workout (e.g., informs pace, effort, and power available). Power exerted can be measured in watts (1001). Embodiments of the present technology, comprising one or more software applications and a computer vision device, can compute power exerted by a user when performing an exercising movement (907. 1003). Data can be collected about a user physical attributes (e.g., weight, height, etc.). Physical attributes can be estimated or assumed. The force when moving body parts and objects held by a user can be estimated. Objects can be items held or worn by a user, such as dumbbells or a weight vest. Pose estimation can be performed on a stream of camera images. A Gross power absorbed (GPA) and gross power released (GPR) formula can be applied for a given feature or multiple features by tracking the movement of features or multiple features, estimating force (i.e., multiplying the estimated mass of a given feature or multiple features by their acceleration), multiplying the result by the displacement of the features or multiple features, and dividing the result by the delta time for a feature or multiple features to travel during the tracked movement. The wight of body parts and objects, or a user weight force, can be factored into the computations. Embodiments of the present technology can give the result of total power generated when a user engages a muscle or muscle group, for example, by estimating mass and computing acceleration, displacement, and time taken for a leg to move through the upward motion of a leg lift against the legs weight and applying a GPA and GPR estimation formula. GPR can give the total power generated when a user releases a muscle, for example, by estimating mass and computing acceleration, displacement, and time taken for a leg (i.e., represented by multiple features), factoring in its weight, to move through the downward motion of a leg lift. Power exerted can inform how to plan for future workouts by measuring a user's functional threshold power (1102) (FTP) and functional reserve capacity (1101) (FRC). FTP can be computed by adding GPA and GPR, factoring in a user weight force, over time and by measuring the average number of watts (1001) a user can sustain prior to experiencing fatigue or FTP (1102). FRC shows how much power remains (1101) in a user session prior to reaching the FTP. Instructions that maximize power exertion over time can adjust instructions to a user to maximize time spend in the FRC zone (1101) and prevent a user from falling below a user FTP (1102). The more time GPA and GPR are calculated over time for a user, the more accurate FTP and FRC can become. A machine learning model can be trained to track a subject with GPA, GPR, FTP and FRC over time for one or more users to enhance instruction to a user.

Embodiments to enhance fitness instruction, feedback, and tracking can be extended by those skilled in the art. Estimating calories burned (908), for example, can be enhanced with embodiments of the present technology comprising a multiplier of the basal metabolic rate by activity level as expressed in TuT or power exerted as compared to a repetition counter that makes broader generalizations about the quality of an exercise movement. The advantage over current exercise technology is that the disclosed invention can provide evaluations of how the full body movement of a user performs with affordable monocular vision hardware while performing an exercise movement rather than relying on non-vision based indicators like heart rate, oxygen sensors, and patterns as done with step counters, or less efficient and more costly depth sensor arrays that can have a higher margin for error, or physics engines that can have costly computational requirements.

Embodiments of the present technology fulfill a need for a method to personalize exercise instruction. It can adjust instructions based on a user ability, performance during an exercise, response to feedback, or past performance. Such a method would open exercise instruction to a wide range of people, provide more reliable exercise guidance and increase to the chances of exercise instruction providing benefits. Such a method would reduce the chances of injury and improve health, which would lower healthcare costs.

Embodiments of the present technology, comprising a predictive model, can enhance exercise instruction and feedback. Novelties of the present technology can use machine learning to predict exercise outcomes to enhance instructions or feedback. Embodiments of the present technology, which can be realized by one or more software applications and a computer vision device, can modify instructional content as a user is performing an exercise. Embodiments of the present invention can modify future instructions, or which instructional videos are presented to a user, based on a user performance and goals. For example, if a goal is set to achieve a 100% form efficacy, and a user has poor form, successive exercises can be reduced until improved form is exhibited (e.g., if a set of 10 squats are instructed and a user completes 10 of 10 squats with each squat completed earning a form score of 50 out of 100, the user form score for the set of squats can be 500 out of 1000, and can be labeled with 50% form efficacy. If the next leg exercise in the instructional video is a set of 10 jumps, instead of instructing a user to perform 10 jumps, the instructional video can instruct a user to perform 6 jumps, intending to improve form efficacy. If the user performs all six jumps, and the user completes 4 jumps with a form score of 100 out of 100 and 2 jumps with a form score of 25 out of 100, the user performance for the set of jumps can be 450 out of 600, and can be labeled with 75% form efficacy. If the next leg exercise in the instruction video is a set of 10 lunges, instead of instructing a user to perform 10 lunges, the instructional video can instruct a user to perform 5 lunges, etc.). Labels can be used to train a machine learning algorithm to inform a computer on how to adjust instruction based on user performance.

Embodiments of the present technology can provide exercise instruction to a user based on one or more performance goals or indicators, or a combination of such, such as exercise intensity, form, TuT, power, strength, weight lifted, calories burned, or how feedback has resulted on a user in the past or how feedback has resulted for an array of users in the past to enhance methods to provide instruction over time. A machine learning platform can save how one or more users' performance has responded to different instruction and feedback, training the computer to identify and label general patterns amongst one or more users. Labeled patterns can be segmented. They can be segmented based on performance indicators (e.g., power, TuT, intensity, correctness, score), response to feedback or instructions, or attributes about one or more users like body composition (e.g., strength, height, weight, age, etc.). The predictions can be used to improve instructions or feedback given to users or user segments.

Embodiments of the present technology, which can be realized by one or more software applications and a computer vision device, can adapt to how a user responds to instruction and feedback to continuously improve the delivery of exercise training services. It can build on learnings from an array of users to amplify these improvements in fitness training services. This dynamic and continually improving delivery of exercise instruction offers the advantage of not being limited to static instruction and feedback. It is designed to provide exercise instruction that has been proven successful over continued use of a single user or an ever-expanding sample of users, so that it is not limited to knowledge of an individual trainer or coach. It offers these advantages and can offer speed, cost, and accuracy improvements when compared to expensive physics engines or the reliance on depth sensors. It offers these advantages without a device that is portable and can be used in a wide range of places (e.g., home, office, hotel, etc.).

Embodiments of the present technology can help motivate a user to better achieve their fitness goals. It can hold users accountable to complete workouts, follow an exercise plan, or increase performance without the need for the physical presence of a fitness partner, coach, or trainer. Embodiments of the present invention can create rewards for exercising that are the property of a user due to embodiments comprising a computer vision device, cryptographic hashing, and a distributed ledger network to hold users accountable to the completion of exercise challenges.

Embodiments of the present technology, realized by cryptographic hashing software and a computer vision device, can enhance fitness motivation with rewards. Rewards for completing an exercise challenge may be audio or video affirmation of a user's success or rewards may be digital assets that are sent as property of a user, such as a non-fungible token or coin recorded in a distributed ledger network. Non-fungible tokens can be embodied as graphical artifacts, digital skins, and certificates to reclaim merchandise like exercise equipment. Coins may be called by a different name, such as points, tokens, fitness currency, or digital currency. If a user is rewarded with a coin, a coin may be immaterial, or may be redeemed for products such as gift cards, gift certificates, cash rewards, checks, discounts on products, merchandise, or for digital products like video content or instructional classes. Digital assets can be the property of a user, and be sold, transferred, or traded by a user. Coins can be sold in a marketplace, redeemed for products such as gift cards, gift certificates, cash, checks, discounts on products, merchandise, or for digital products like video content or instructional classes. Coins can be minted. Coins can be issued on an existing distributed ledger network, a decentralized application over an existing distributed ledger network, or a new distributed ledger network can be created. Digital assets can be earned as a reward for completing an exercise movement, exercise challenge, challenge, can be staked on a distributed ledger network to earn additional coins, or can be earned for contributing computational resources to the operation and health of a distributed ledger network. Coins can be minted in exchange for completing an exercise challenge, staking coins on a distributed ledger network, or contributing computation resources to the operation and health of a distributed ledger network.

Embodiments of the present technology, comprising cryptographic hashing and a network of nodes, can enhance exercise motivation with rewards. A user can receive a reward based on exercising, measured by performance indicators, ranking on a leaderboard, movement between or within ranked segments of users, on the quantity or quality of exercise progress, or completing an exercise challenge (e.g., user completes record number of workouts, user completes workout at a higher performance level than previous performance levels for the same workout, user wins a competition amongst peers, etc.). An exercise challenge can be created on a distributed ledger network or decentralized application built on an existing distributed ledger network. An exercise challenge can correspond to exercise or fitness goals. An exercise challenge, comprising exercise movements, performance goals, and a coin reward, can be completed in exchange for a reward. It can comprise a user staking coins to participate in an exercise challenge or a user staking no coins to participate in an exercise challenge. Through one or more software applications, a human pose can be estimated. A user can be instructed to complete an exercise challenge comprising one or more exercise movements and a token reward. Performance tracking data can be computed. Data from exercise performance metrics can be placed into a text readable form (1204). A hash function can turn one or more sets of exercise movements into a hash or a summary hash of one or more exercise movements (1205).

Embodiments of the present technology can validate or authenticate a user has completed an exercise challenge (FIG. 12). When a user completes an exercise challenge, a message (1207) can be broadcasted to a distributed network (1202) comprising data from the exercise challenge (1204) that can be cryptographically hashed (1205) and signed by a user private key (1206). A node can stake coins to participate in the reward validation process and claim a coin fee in exchange for validation services. At least one node (1203) can validate a user has completed an exercise challenge and can respond to a user broadcast with a coin reward challenge message (1211) comprising movement instructions (1208) to a user that can be cryptographically hashed (1209) and signed by a node (1210). A user (204) can complete the movement and broadcast a message (1215) to the network, a message comprising data (1213) from the movements completing a coin reward challenge (1208) that can be hashed (1214) and signed by a user private key (1206). A node (1203) can verify a user has completed a coin reward challenge (1213) and exercise challenge (1204) signing (1210) a hash (1216) and can broadcast a reward transaction (1218) with a coin award addressed to a user. A reward transaction (1218), comprising data from an exercise challenge (1204), a coin reward challenge (1213) and coin reward, signed by a node key (1210), can be used to create a digital signature of a reward transaction that can be broadcasted (1218). A user can receive the message (1218) for a reward transaction and sign it (1219) with a user public key (1220) to create a hash (1221). A user can verify the hash of a reward transaction (1221) is valid. A user can verify a hash of a reward transaction (1221) is valid by comparing it to hash (1223) comprising data from an exercise challenge (2014), coin reward challenge (1208), coin reward, and coin reward movement (1213). A result of matching the hash produced (1223) with a signed reward transaction from a node (1221) can verify the transaction (1230) as valid. One or more nodes can repeat the validation process to authenticate a coin reward transaction, comprising, hashing (1219) a user public key (1220) with a signed reward transaction (1218) to produce a (1221) hash of the data; hashing (1222) movement and reward data (1204, 1208, 1213) to create a hash of the data (1223); evaluating whether the hashes match (1230). If a number of nodes (e.g., majority) reproduce a hash (1223) that matches the user generated hash (1221), the transaction can be saved into an immutable block in a distributed ledger network, thereby recording the transaction and ownership of respective coins distributed by a coin reward transaction. Coins can be saved on a distributed ledger network as property owned by a user in exchange for completing an exercise challenge and coins can be saved on a distributed ledger network as property owned by one or more nodes for facilitating a transaction. A node can stake coins to participate in the reward validation process and claim a coin fee in exchange for validation services. If a number of nodes (e.g., majority) cannot validate the coin reward transaction, it can expose a user and nodes to a risk of losing coins staked as a penalty for attempting to defraud the coin reward process. Nodes that validate transactions can be separated into validation cohorts.

Embodiments of the present technology, comprising a method for validating exercise transactions, can validate transaction on a distributed ledger network with a randomized cohort system. At the beginning of each new block on a distributed ledger network, three or more random validation cohorts can be created. Each member within a cohort can be assigned based on a user or node public key. Once a user completes an exercise challenge, a message can be broadcasted to a distributed network comprising data from the exercise challenge signed by a user private key. At least one node from a user validation cohort can choose to validate a user has completed an exercise challenge by sending a user a coin reward challenge comprising instructions to a user.

Embodiments of the present technology are not limited to the validation methods disclosed. As a person skilled in the art may attest, embodiments of the present technology can extend methods disclosed. Embodiments of the present technology can include a validation period at the end of a block cycle. During a validation period, transactions can be suspended. Transaction attempted past the validation cycle timestamp can request a coin reward challenge on a succeeding block. The transaction suspension period can give time for nodes to catch up in validation, particularly if the network does not have enough active nodes to validate reward challenge transactions.

The novel method of enhancing exercise motivation comprising a method to cryptographically hash computer vision data of exercise movements for completing an exercise challenge can be realized with alternative embodiments for rewarding an exercise movement. For example, one or more users can enter into an agreement to complete an exercise challenge with a smart contract. The smart contract can contain an exercise challenge, requirements to complete an exercise challenge (e.g., performance indicators, leaderboard ranking, accomplishing goals, setting records) and a reward assigned to the challenge, can be recorded in a block comprising transactions on a distributed ledger. As a user exercises in front of a computer vision device, data collected about the competition or progress toward the completion of an exercise challenge can be computed into a text readable form. A hashing algorithm can compute one or more sets of data into a hash signed by a private key. A hashing algorithm can compute one or more sets of data into a hash signed by a private key and one or more public keys. When the exercise challenge is completed, one or more hashes corresponding to the completion of the exercise challenge can complete the execution of a smart contract. A transaction can give the reward to one or more users who completed an exercise challenge. The transaction can be saved to the distributed ledger network and be made immutable. Embodiments of the present technology comprising cryptographic hashing and a network of computer vision devices to distribute exercise can be extended to with alternative reward protocols.

Embodiments of the present technology can distribute resources (e.g., sharing processing time, hard drive space, and exercise videos for the benefit of a network) amongst a network of computer vision devices to enhance exercise instruction, such as sharing instructional video content, creating and executing smart contracts, and validating transactions. Nodes on a distributed ledger network that distribute resources can be rewarded for their contributions to the network with a proof of stake mechanism, proof of completing exercise challenges, or proof of sharing resources. Proof mechanisms can be combined, such as requiring a proof of completing an exercise challenge as a pre-request to the coins available to stake with a proof of stake mechanism or can require a user to staking coins before entering into an exercise challenge.

As a person skilled in the art may attest, embodiments of the present technology extend beyond exercise and into movement. An exercise challenge can relate to movement such as dance. It can embody mechanisms to capture full body movement and motivate activity with a reward, that through the novel use of cryptographic hashing a distributed ledger network, and validation, can be comprised. Embodiments of the present invention can advantageously hold users accountable to complete workouts, follow an exercise plan, or increase performance without the need for the physical presence of a fitness partner, coach, or trainer. Embodiments of the present invention can provide valuable rewards due to the novel process of computing data about human movement with a compute vision device, validating movements, and holding users accountable to the completion of exercise challenges. The novel systems and methods disclosed can provide opportunity for more meaningful rewards that motivate healthier lifestyles, better fitness, and reducing healthcare costs.

What is claimed:

1. A computer vision exercise device, comprising:
at least one camera coupled to the device, wherein a camera is configured to capture the movement of a user as a user exercises in front of the device;
at least one controller coupled to the device, wherein the controller is configured to navigate and initialize exercise instructions; and
a computer-readable storage medium comprising executable instructions that, when executed, cause a computer to access information coupled to the device comprising exercise instructions; project exercise instructions to user on a connected display; initialize one or more machine learning models; track exercise movement of at least one user with a vision sensor; perform processing on sensor data to normalize data; perform human pose estimation on data collected about a user movement; compare a user pose with a fitness instructor pose, wherein the difference between one or more features on a user pose and the fitness instructor pose are computed; and communicate at least one piece of information regarded a user exercise performance to a user through a connected display.

2. The computer exercise vision exercise device according to claim 1, comprising at least one sensor attached to a user body that detects the movement of a user body to improve human pose estimation.

3. The computer vision exercise device according to claim 1, comprising at least one sensor within the device to estimate the orientation of the camera within a device in relation to a user performing an exercise movement.

4. The computer vision exercise device according to claim 1, comprising a material concealing a camera lens during camera operation so that a user cannot see the camera lens when performing an exercise movement.

5. The computer vision exercise device according to claim 1, comprising a resistance hinge fixed to the bottom of the device that can mount the device to the top of a display or when fully closed can be used as a stand to place the device on a flat surface.

6. The computer vision exercise device according to claim 1, comprising more than one device that are nodes in a network which together act as a server to one or more devices that act as a receiver.

7. The computer vision exercise device according to claim 1, comprising at least one neural processor to accelerate machine learning operations.

8. A virtual exercise system comprising:
an exercise environment with at least one fitness instructor, instructing exercise to a user;
a simulation of a user within the exercise environment;
a control system where a camera captures movement of a user body to control a simulation of a user displayed in the exercise environment;
a dashboard that displays information about a user body movement as it relates to exercise performance;
a virtual exercise simulator comprising a user simulation within an exercise environment, wherein the movement of a user simulation is controlled by the body movement a user, wherein information about a user exercise performance is shown on a dashboard to a connected display to a user.

9. The system according to claim 8, wherein an instructional simulation is overlaid on a user simulation to visualize exercise instructions to a user.

10. The system according to claim 8, wherein an avatar with an adjustable likeness can represent a user simulation.

11. The system according to claim 8, wherein a remote user is projected as a simulation in the exercise environment that moves synchronously to the movement of a remote user.

12. The system according to claim 8, wherein a computer vision exercise device is used to control a user movement and project the simulation onto a user display.

13. A method for predicting the third dimension during movement, comprising:
capturing camera images of a user;
estimating a two-dimensional pose when a user is in a baseline posture and at least one dimension of at least one body feature;
instructing a user to perform a movement;
identifying when the estimation of at least one dimension of at least one body feature differs in a baseline posture than the estimation of a user posture during a movement; and
applying an algorithm, comprising kinematics, user orientation with respect to a camera and a geometric theorem, to predict the third dimensional movement of at least one body features.

14. The method according to claim 13, wherein at least one IMU is placed within a computer vision device to determine the orientation of a camera capturing images of a user to reduce distortion introduced by monocular vision and a user orientation to a camera.

15. The method according to claim 13, wherein at least one IMU is attached to a user to reduce distortion introduced by monocular vision and a user orientation to a camera.

16. The method according to claim 13, wherein a computer vision device is configured to capture a camera images of a user body.

17. The method according to claim 13, wherein data is entered into a computer by a user about a user body dimensions to inform a user baseline posture.

18. The method according to claim 13, wherein a software application can estimate user height or body dimensions to inform a user baseline posture.

19. A method for enhancing exercise instruction, comprising:
capturing camera images of a fitness instructor performing at least one exercise movement;
performing pose estimation on a fitness instructor performing an exercise movement;
extracting pose estimation features to train a machine learning model to recognize an instructional pattern from at least one exercise movement;
configuring a computer vision device to capture a camera stream of a user performing an exercise movement;
instructing a user to perform an exercise movement in front of a computer vision device, extracting features from pose estimation performed on camera images of a user, comparing features of a movement of a user to an instructional pattern; and
providing at least one piece of feedback to a user.

20. The method according to claim 19, wherein an intensity score is computed comprising the delta value between at least one estimated feature extracted during an instructional movement and at least one feature extracted during the movement of a user when performing an exercise.

21. The method according to claim 14, wherein a form score is computed comprising the delta value between at least one feature estimated during an instructional movement of an instructional pattern and at least one feature estimated during the movement of a user when performing an exercise.

22. The method according to claim 14, wherein exercise instructions are adjusted based on at least one exercise performance metric.

23. The method according to claim 14, wherein time under tension for at least one muscle is calculated.

24. The method according to claim 14, wherein a predictive model is trained to compute feedback for a user performing an exercise movement.

25. The method according to claim 14, wherein features, angles and relative distance amongst body features or points with respect to the time it takes to complete an exercise movement by a fitness instructor and user are compared to compute at least one exercise performance metric.

26. The method according to claim 14, wherein features, angles and relative distance amongst body features or points with respect to the time it takes to complete an exercise movement by a fitness instructor and user are compared to compute exercise at least one piece of exercise feedback.

27. A method for estimating power generated from an exercise movement, comprising:
capturing camera images of a user performing an exercise movement;
estimating a human pose of a user performing an exercise movement;
computing the force applied by a user and a user appendages; and
tracking an exercise movement through a stream of camera images to estimate the energy generated from the movement of at least one body part or appendage.

28. The method according to claim 27, wherein a user power reserve can be computed to instruct a user on how much effort to exert during an exercise activity.

29. The method according to claim 27, wherein the formula to compute energy generated factors weight force of a user into gross power absorbed and gross power released.

30. The method according to claim 27, wherein the formula to compute energy generated factors weight force of a user and objects held by a user into gross power absorbed and gross power released.

31. A method for enhancing fitness motivation, comprising:
creating an exercise challenge with a reward for completing at least one exercise movement;
capturing camera images of a user performing an exercise movement;
computing a cryptographic hash of a user performing an exercise movement to complete an exercise challenge;
verifying an exercise challenge was completed;
providing a reward to a user for completing an exercise challenge; and
configuring a network of nodes to record rewards given to a user on a distributed ledger.

32. The method according to claim 30, wherein one or more users contributing at least one cryptographic hash of an exercise movement completes a smart contract.

33. The method according to claim 30, wherein a network of more than one device is configured to distribute computational resources to support the operation of a distributed ledger network.

34. The method according to claim 30, wherein exercise movement data computed into a cryptographic hash is verified by one or more nodes on a distributed ledger network to authenticate the completion of an exercise challenge.

35. The method according to claim 30, wherein a movement request is created by nodes on a distributed ledger network to authenticate a user has completed an exercise movement or challenge.

36. The method according to claim 30, wherein human pose estimation of a user is compared to an exercise instructor to measure the success of completing an exercise challenge.

37. The method according to claim 30, wherein cohorts participate in a consensus mechanism for validating transactions.

38. The method according to claim 30, wherein a staking mechanism is required for a user to participate in an exercise challenge.
